(19) United States
(12) Patent Application Publication
(10) Pub. No.: US 2026/0014021 A1
(43) Pub. Date: Jan. 15, 2026

(54) SURGICAL METHOD FOR INSERTION OF IMPLANT DEVICE FOR EYE DISEASE

(71) Applicant: MICROT INC., Seoul (KR)

(72) Inventors: Eunsuk LEE, Seongnam-si (KR); Yyejin SONG, Seoul (KR)

(21) Appl. No.: 18/799,098

(22) Filed: Aug. 9, 2024

(30) Foreign Application Priority Data
Jul. 11, 2024 (KR) 10-2024-0091561

(51) Int. Cl. A61F 9/007

(52) U.S. Cl. CPC A61F 9/00781

(57) ABSTRACT

A surgical method for insertion of an implant for eye diseases may include: cutting a portion of a sclera of an eyeball to form a scleral flap to be lifted up from the eyeball; inserting an end of a tube including a hole for aqueous humor drainage from an anterior chamber of the eyeball into the eyeball through an area beneath the scleral flap; and covering the tube exposed to outside of the eyeball with the scleral flap. Herein, an opposite end of the tube is not inserted into the eyeball. Further, covering with the scleral flap comprises the step of placing the scleral flap on the tube without suturing of the scleral flap that leads to blockage or narrowing of a route of the aqueous humor drainage through the tube exposed to the outside of the eyeball.

### CROSS-REFERENCE TO RELATED APPLICATION

This application claims priority to Korean Patent Application No. 10-2024-0091561, filed on July 11, 2024, and all the benefits accruing therefrom under 35 U.S.C. §119, the contents of which in its entirety are herein incorporated by reference.

### BACKGROUND

### Field

The present disclosure relates to a surgical method for insertion of an implant for eye diseases, and more particularly, to a method for inserting an implant for eye diseases into an eyeball, including forming a scleral flap and inserting the implant under the scleral flap to avoid direct contact between a proximal end of the implant, exposed to the outside of the sclera, and the sclera and the Tenon's capsule in order to prevent wound dehiscence.

### Description of the Related Art

For glaucoma patients who have difficulties in adjusting the intraocular pressure despite the use of drugs for lowering the intraocular pressure, the intraocular pressure is lowered by bypassing the aqueous humor from the anterior chamber of the eye under the conjunctiva outside of the eye. One of glaucoma filtration surgeries used to create a pathway or a fistula for aqueous humor drainage, trabeculectomy fails to adjust the intraocular pressure due to the reduced drainage volume of the aqueous humor caused by blockage of the pathway after surgery. When a glaucoma filtration surgery is performed again due to an unsuccessful outcome of the previous surgery, a pathway is blocked more frequency after surgery and the surgery success rate is low.

Additionally, in the case of the type of glaucoma that is difficult to treat such as neovascular glaucoma or secondary glaucoma caused by uveitis, the outcomes of trabeculectomy are not pretty good due to frequent pathway blockage after surgery. In the case of eyes that have failed with glaucoma filtration surgery or glaucoma that is difficult to treat, surgery involving inserting a glaucoma implant device (glaucoma implant surgery) is performed to prevent pathway blockage in order to increase the surgery success rate. To date, glaucoma implants are used as an alternative to trabeculectomy especially in some types of glaucoma that are difficult to treat because they effectively lower the intraocular pressure and show predictable post-operative clinical outcomes according to the preset inside diameter of the tube.

However, the existing glaucoma implants used in glaucoma implant surgery may cause many problems and complications, for example, difficulties in surgery due to the relatively large size, post-operative exposure, inflammation, eye movement disorders induced by the large body and double vision (diplopia). Accordingly, recently, small glaucoma implant instruments for Minimally-Invasive Glaucoma Surgery (MIGS) are developed to lower the intraocular pressure relatively easily and reduce side effects that occur after surgery due to the large size.

MIGS involves inserting the tube having micrometer-scale inside diameter into the anterior chamber of the eye to enhance the drainage of the aqueous humor from the anterior chamber, and when the proximal end of the tube exposed to the outside of the eyeball comes into direct contact with the sclera and the Tenon’s capsule, wound dehiscence may occur. Additionally, the tube may be blocked at the end of the tube inserted into the eyeball, the sutured surface of the tube and the aqueous humor outlet of the exposed tube due to fibrosis of the tissue around the eye. When the aqueous humor outlet of the tube is blocked, the aqueous humor does not drain through the MIGS tube and the intraocular pressure in the glaucoma patient rises again. To solve this problem, re-surgery is needed to dissect the fibrotic part or insert the implant again. In particular, when the tube is sutured to the eyeball, the aqueous humor drains through the tube in the early stage but as time goes by, there is a high risk that the outlet will be blocked.

Additionally, MIGS involves inserting an intraluminal stent into the tube to adjust the drainage volume of the aqueous humor, and the intraluminal stent is used to prevent the initial intraocular pressure reduction after the implant surgery and needs to be removed after a predetermined time (for example, about 4 weeks to 16 weeks). However, in case the intraluminal stent is not properly secured in the surgical procedure of inserting the implant, when the patient rubs the eye, the intraluminal stent may slip, or the intraluminal stent may be buried in the conjunctiva, which makes it difficult to remove. When the intraluminal stent is buried in the conjunctiva, the intraluminal stent may be removed through a needle, but a bleb formed during the drainage of the aqueous humor unavoidably breaks in the removal process, affecting the aqueous humor drainage.

### SUMMARY

According to an aspect of the present disclosure, a surgical method for insertion of an implant for eye diseases is provided, in which a scleral flap is formed and the implant for eye diseases is inserted into the eyeball under the scleral flap. This may prevent direct contact between the proximal end of the implant, exposed to the outside of the sclera, and the sclera and the Tenon’s capsule. Further, suturing that leads to blockage or narrowing of the route of aqueous humor drainage is not performed to solve the conventional problem, and an intraluminal stent inserted into the implant tube is properly secured.

A surgical method for insertion of an implant for eye diseases according to an aspect of the present disclosure comprises: cutting a portion of a sclera of an eyeball to form a scleral flap to be lifted up from the eyeball; inserting an end of a tube including a hole for aqueous humor drainage from an anterior chamber of the eyeball into the eyeball through an area beneath the scleral flap; and covering the tube exposed to outside of the eyeball with the scleral flap.

Herein, an opposite end of the tube is not inserted into the eyeball, and covering with the scleral flap comprises placing the scleral flap on the tube without suturing of the scleral flap that leads to blockage or narrowing of a route of the aqueous humor drainage through the tube exposed to the outside of the eyeball.

According to an embodiment, the covering with the scleral flap comprises: folding the scleral flap such that a first side of the scleral flap covering the tube exposed to the outside of the eyeball is lifted up; and suturing together a portion of the scleral flap and the eyeball or other portion of the scleral flap to prevent the folded scleral flap from being unfolded.

According to an embodiment, the suturing comprises suturing together the first side of the scleral flap and a second side of the scleral flap opposite the first side, wherein an aqueous humor outlet of the tube is exposed, but not covered by the scleral flap.

According to an embodiment, the tube further includes an intraluminal stent that is inserted into the hole and exposed through the opposite end of the tube, and the surgical method for insertion of the implant for eye diseases further comprises allowing, in at least part, the intraluminal stent exposed through the opposite end of the tube to pass through the conjunctiva of the eyeball to secure the intraluminal stent.

According to an embodiment, the securing the intraluminal stent comprises exposing a portion of the intraluminal stent to outside of the conjunctiva of the eyeball.

According to an embodiment, the securing the intraluminal stent further comprises inserting a tip of the intraluminal stent into the conjunctiva of the eyeball with the portion of the intraluminal stent staying outside of the conjunctiva of the eyeball.

According to an embodiment, the securing the intraluminal stent further comprises suturing the intraluminal stent to the conjunctiva at a location between an area in which the portion of the intraluminal stent passes through the conjunctiva and a boundary of the scleral flap in order to secure the intraluminal stent.

According to an embodiment, the exposing the portion of the intraluminal stent to the outside of the conjunctiva of the eyeball comprises: inserting a syringe needle into the conjunctiva from the outside of the conjunctiva of the eyeball; inserting the portion of the intraluminal stent into the syringe needle; and removing the syringe needle from the conjunctiva to move the portion of the intraluminal stent out of the conjunctiva.

According to an embodiment, the securing the intraluminal stent further comprises removing, in at least part, the intraluminal stent exposed to the outside of the conjunctiva of the eyeball.

According to an embodiment, the surgical method for insertion of the implant for eye diseases further comprises: before inserting into the eyeball, treating the area beneath the scleral flap with an antifibrotic agent.

According to an embodiment, the tube includes at least one wing extended in a direction that is different from a length direction of the tube and configured to limit an insertion location of the tube, and the inserting into the eyeball comprises adjusting the insertion location of the tube to prevent the at least one wing from being inserted into the eyeball.

According to an embodiment, the inserting into the eyeball comprises: forming a hole passing through the sclera at a location away from a connected part of the scleral flap to the sclera by a preset distance; and inserting the end of the tube into the anterior chamber of the eyeball through the hole.

In the conventional implant surgical method for eye diseases without forming the scleral flap, the proximal end of the implant tube exposed to the outside of the eyeball may come into direct contact with the sclera and the Tenon’s capsule, causing wound dehiscence, and the aqueous humor outlet at the tip of the tube may be blocked by fibrosis of the Tenon’s tissue. Additionally, when antifibrotic therapy using mitomycin-C (MMC) is performed in the absence of the scleral flap, the Tenon’s capsule tissue may become weak, and when it comes into contact with the implant tube, the tube may be exposed.

In contrast, by the surgical method for insertion of the implant for eye diseases according to an aspect of the present disclosure, the tube of the implant is inserted into the eyeball under the scleral flap, and the tube is positioned beneath the scleral flap with no sutures that block or narrow the route of aqueous humor drainage through the tube at the proximal end of the tube to drain the aqueous humor, thereby preventing direct contact between the tube and the sclera and the Tenon’s capsule and prevent the aqueous humor outlet of the tube from being blocked by fibrosis.

With the surgical method for insertion of the implant for eye diseases according to an aspect of the present disclosure, there is no risk that the route of aqueous humor drainage will be blocked by fibrosis at the tip of the insertion part of the tube or the sutured surface of the tube, and compared to the prior art, it may be possible to enhance surgery prognosis prediction by preventing fibrosis near the conjunctiva and improve long-term prognosis by controlling fibrosis.

Additionally, by the surgical method for insertion of the implant for eye diseases according to an aspect of the present disclosure, it may be possible to achieve stable drainage of the aqueous humor through the combination of the intraluminal stent inserted into the tube of the implant. In this instance, because the position of the intraluminal stent may be fixed by allowing the tip of the intraluminal stent to pass through the conjunctiva or inserting it into the conjunctiva, when removing the intraluminal stent after a predetermined time has passed since implantation, it may be possible to easily remove without affecting the aqueous humor drainage through the implant.

Further, by the surgical method for insertion of the implant for eye diseases according to an aspect of the present disclosure, the tube of the implant device may be buried in the sclera to prevent the exposure of the tube, thereby achieving more efficient surgery and preventing complications.

### BRIEF DESCRIPTION OF THE DRAWINGS

FIGS. 1 and 2 are conceptual views showing an implant device for eye diseases inserted into an eyeball according to an embodiment.

FIG. 3 is a flowchart showing each step of a surgical method for insertion of an implant for eye diseases according to an embodiment.

FIGS. 4A to 4C are conceptual views showing a process of forming a scleral flap in a surgical method for insertion of an implant for eye diseases according to an embodiment.

FIGS. 5A to 5D are conceptual views showing a process of inserting an implant under a scleral flap in a surgical method for insertion of the implant for eye diseases according to an embodiment.

FIGS. 6A and 6B are conceptual views showing space formation between an aqueous humor outlet of an implant and conjunctiva in a surgical method for insertion of the implant for eye diseases according to an embodiment.

FIGS. 7A and 7B are conceptual views showing a scleral flap folded to prevent an aqueous humor outlet of an implant from being covered, in a surgical method for insertion of the implant for eye diseases according to an embodiment.

FIGS. 8A and 8B are conceptual views showing that an intraluminal stent exposed to the outside of an eyeball is secured using conjunctiva in a surgical method for insertion of an implant for eye diseases according to an embodiment.

### DETAILED DESCRIPTION

The terms as used herein will be briefly described, and the present disclosure will be described in detail.

The terms as used herein are general terms selected as those being now used as widely as possible taking into account the functions in the present disclosure, but may vary depending on the intention of those skilled in the art or the convention or the emergence of new technology. Additionally, in certain cases, there may be terms arbitrarily selected by the applicant, and in this case, the meaning will be described in detail in the corresponding description part of the specification. Accordingly, the terms used herein should be defined based on the meaning of the terms and the entire description of the present disclosure rather than simply the name of the terms.

The terms "comprise" and "include" when used in this specification, specify the presence of stated element but do not preclude the presence or addition of one or more other elements unless the context clearly indicates otherwise. Additionally, when an element is referred to as being "connected to" another element, it can be "directly connected to" the other element and "intervening elements" may be present.

Hereinafter, the embodiment of the present disclosure will be described in sufficient detail for persons having ordinary skill in the technical field pertaining to the present disclosure to easily carry out the invention with reference to the accompanying drawings. However, the present disclosure may be embodied in many different forms and is not limited to the disclosed embodiment. Additionally, in the drawings, for clarity purposes, irrelevant illustration to the description is omitted, and similar reference signs are affixed to similar elements throughout the specification.

Hereinafter, the present disclosure will be described in detail with reference to the accompanying drawings.

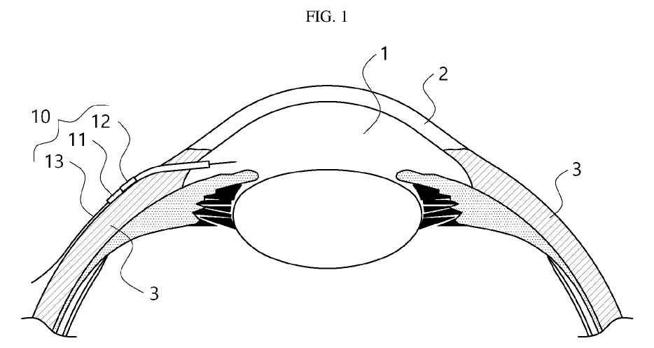

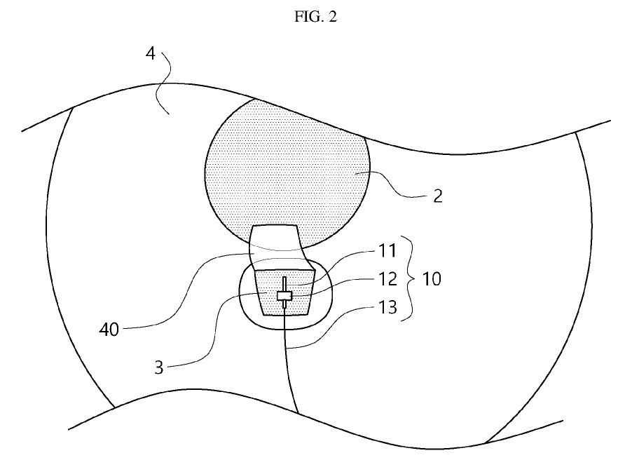

FIGS. 1 and 2 are conceptual views illustrating an implant device used in a surgical method for insertion of an implant for eye diseases according to an embodiment and the insertion of the device into the eye.

Referring to FIGS. 1 and 2, at least part of the implant device 10 for eye diseases is inserted into the sclera 3 of the eyeball. In the specification, the implant device 10 used in the surgical method according to the embodiments will be described by taking an implant for glaucoma treatment as an example. However, the implant device 10 for eye diseases used in the surgical method according to the embodiments of the present disclosure may be used to treat ocular hypertension or its consequential eye diseases or alleviate symptoms.

For use in the treatment of glaucoma, the implant device 10 is used to control the intraocular pressure by adjusting the drainage volume of the aqueous humor produced in the anterior chamber 1 located in front of the crystalline lens and under the cornea 2 in the eye, and plays a role in preventing optic nerve damage caused by ocular hypertension due to eye diseases.

The eye disease as used herein may include glaucoma caused by increased intraocular pressure, and the glaucoma may include congenital glaucoma, traumatic glaucoma, glaucoma suspect, high tension glaucoma, primary open angle glaucoma, normal tension glaucoma, capsular glaucoma with pseudoexfoliation of lens, chronic simple glaucoma, low tension glaucoma, pigmentary glaucoma, primary angle closure glaucoma, acute angle closure glaucoma, chronic angle closure glaucoma, intermittent angle closure glaucoma, glaucoma caused by an injury to the eye, glaucoma caused by inflammation in the eye, drug-induced glaucoma, neovascular glaucoma or secondary glaucoma caused by uveitis, but is not limited thereto.

In an embodiment, the implant device 10 may include a tube 11 that can be applied to Minimally-Invasive Glaucoma Surgery (MIGS), and one end of the tube 11 may be inserted into the anterior chamber 1 of the eyeball and the other end of the tube 11 may be located at the conjunctiva tissue or the Tenon’s tissue. The tube 11 includes a hole in which the aqueous humor in the eye may flow, so that the aqueous humor drains from the anterior chamber 1 of the eyeball through the tube 11.

More specifically, the tube 11 is inserted into the eyeball such that among two ends of the tube 11, the distal end is located within the anterior chamber 1 of the eyeball and the proximal end is located at the conjunctiva tissue or the Tenon’s tissue of the eyeball, in order to drain the aqueous humor produced in the anterior chamber of the eyeball to the conjunctiva tissue or the Tenon’s tissue through the hole in the tube 11. The distal end and the proximal end as used herein are defined according to a direction from an operator who inserts the implant device 10, and among the two ends of the tube 11, the proximal end refers to an end in a direction toward the operator and the distal end refers to an end in a direction toward a patient's eye into which the implant device 10 will be inserted.

In an embodiment, the tube 11 may be made of a biocompatible material, and may include an adaptable material. For example, the tube 11 may be made of silicone or other silicone-based material, polytetrafluoroethylene (PTFE), a urethane-based material such as polycarbonate or polyurethane (PU), a composite of the silicone-based material and the polyurethane-based material such as silicone-PU, or a biocompatible metal or alloy.

In an embodiment, the tube 11 may include any one of silicone, PTFE, polycarbonate, polyurethane, polyethylene, polypropylene, polyimide, polymethyl methacrylate (PMMA), poly(styrene-b-isobutylene-b-styrene), polyethersulfone, gelatin, stainless steel, titanium and nitinol or a combination thereof, but is not limited thereto.

In an embodiment, the tube 11 may form a curve having a predetermined curvature to prevent damage to the corneal endothelium in the eye. Due to different eyeball sizes of each patient and skill proficiency in tube injection, while the tube is inserted into the anterior chamber of the eyeball, the anterior end of the tube may prick the cornea in the anterior chamber of the eyeball, causing damage, and the cornea damage may cause complications including corneal dysfunction that requires a cornea transplant afterwards. By this embodiment, the tube 11 may be bent with the predetermined curvature corresponding to the curvature of the eye surface so that the movement of the tube may naturally form a curve while the tube is inserted into the anterior chamber of the eyeball.

In an embodiment, the implant device 10 includes a wing 12 coupled to the outer surface of the tube 11 and at least part of the wing 12 has a larger cross section than the diameter of the tube 11. As a result, a portion of the wing 12 protrudes in the lateral direction of the tube 11, that is, a direction that is different from the length direction of the tube 11. Due to the presence of the wing 12 extended in the lateral direction of the tube 11, when the tube 11 is pushed into the sclera 3 of the eyeball, the wing 12 may get stuck in the sclera 3, thereby preventing the tube 11 from being completely inserted into the eyeball. The wing 12 may be formed by coupling or joining at least one member to the surface of the tube 11, or inserting the tube 11 into the member extended in the direction that is different from the length direction of the tube 11.

Referring to FIG. 2, when using the surgical method according to the embodiments, the implant device 10 for eye diseases may be inserted into the sclera 3 through the area beneath the scleral flap 40 formed by dissecting a part of the limbus 4 located around the cornea 2 in the sclera of the eyeball by the operator. After the implant device 10 is inserted, the implant device 10 may be covered by the scleral flap 40 again, so the implant device 10 may be positioned inside the eyeball.

That is, after the limbus 4 of the sclera 3 surrounding the cornea 2 is cut to generate the scleral flap 40, the generated scleral flap 40 may be lifted up to insert the distal end of the tube 11 which in turn, passes through the exposed sclera 3. In this instance, the tube 11 is not completely inserted into the sclera 3 and the proximal end of the tube 11 including the aqueous humor outlet is located outside of the sclera 3. After one end of the tube 11 is inserted into the anterior chamber 1 through the sclera 3, the elevated scleral flap 40 may be lifted down. When the implant device 10 for eye diseases is inserted into the eyeball, the aqueous humor produced in the anterior chamber flows through the tube 11 of the implant device for eye diseases, thereby draining the aqueous humor from the anterior chamber and lowering the intraocular pressure.

In the following description, the implant device 10 or the tube 11 of the implant device 10 inserted into the eyeball represents that at least part of the implant device 10 or the tube 11 of the implant device 10 is located in the sclera 3 of the eyeball. On the contrary, a part of the implant device 10 or the tube 11 of the implant device 10 located outside of the eyeball represents that the corresponding part is located outside of the sclera 3 of the eyeball. In this instance, outside of the eyeball is intended to include being covered by the scleral flap 40 outside of the sclera 3.

The surgical method according to the embodiments designs the insertion location of the implant device 10 and the subsequent surgical procedure so that the proximal end of the tube 11 of the implant device 10 exposed to the outside of the sclera 3 is not completely covered by the scleral flap 40. Additionally, in the surgical method according to the embodiments, through a process of suturing a portion of the scleral flap 40 in the other portion of the scleral flap 40, the eyeball or the implant device 10, the portion of the scleral flap 40 covering the tube 11 exposed to the outside of the sclera 3 may be lifted up (that is, in the outward direction of the eyeball), and it will be described in more detail below.

In an embodiment, the implant device 10 for eye diseases further includes an intraluminal stent 13 inserted into the hole of the tube 11. The intraluminal stent 13 is used to adjust the pressure formed in the anterior chamber through the hole of the tube 11. In an embodiment, the intraluminal stent 13 may be a non-absorbable surgical suture thread, and may be made of, for example, nylon or Prolene, but is not limited thereto.

When the intraluminal stent 13 is inserted into the hole of the tube 11, the flow of aqueous humor is not smooth, resulting in an accumulation of aqueous humor in the anterior chamber of the eye and relatively increased intraocular pressure compared to when the intraluminal stent 13 is absent, and the pressure formed in the anterior chamber as used herein refers to the pressure in the anterior chamber of the eyeball formed at that time. When the intraluminal stent 13 is inserted into the tube 11, then the implant device 10 for eye diseases may be inserted into the eyeball, or after the implant device 10 for eye diseases except the intraluminal stent 13 is inserted into the eye, the intraluminal stent 13 may be inserted into the tube 11.

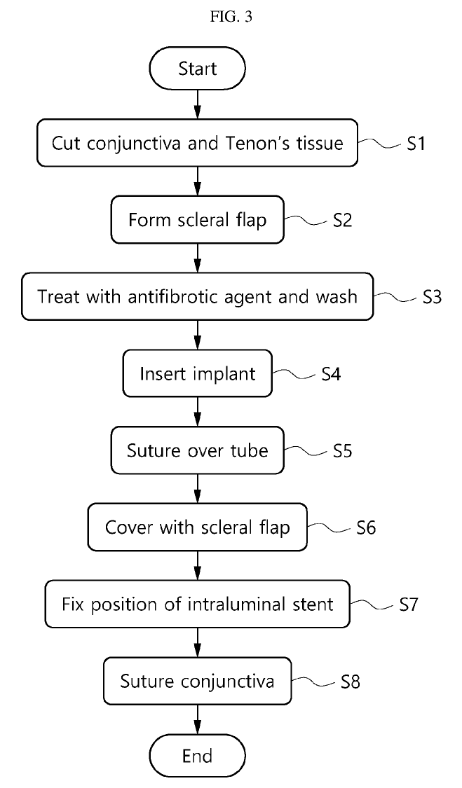

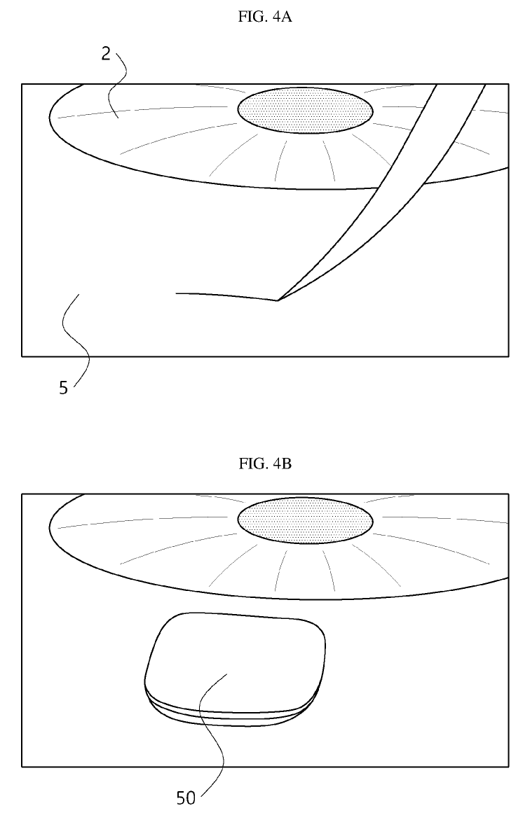

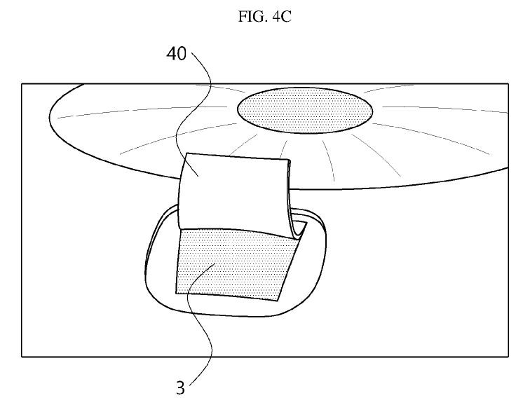

FIG. 3 is a flowchart showing each step of the surgical method for insertion of the implant for eye diseases according to an embodiment, and FIGS. 4A to 4C are conceptual views showing a process of forming the scleral flap in the surgical method for insertion of the implant for eye diseases according to an embodiment.

Referring to FIGS. 3 and 4A, in the surgical method according to this embodiment, first, a part of the limbus surrounding the cornea 2 in the conjunctiva 5 of the eyeball may be cut (S1). In the incision procedure, the Tenon’s tissue located between the conjunctiva 5 and the sclera may be cut together. Although one incision line is shown in the drawing, the conjunctiva 5 of the limbus of the cornea 2 may be cut to a few mm in each of horizontal and vertical lengths. In this instance, the vertical direction refers to an outward direction from the center of the eyeball surface, and the horizontal direction refers to a direction perpendicular to the vertical direction.

Referring to FIGS. 3 and 4B, the conjunctiva may be cut in the horizontal and vertical direction to form an incision 50 including the conjunctiva and the Tenon’s tissue. Subsequently, the conjunctiva and the Tenon’s tissue may be dissected to a constant depth along the edge of the incision 50. For example, the conjunctiva and the Tenon’s tissue may be dissected at 90^{o} to 120^{o} to the depth of from 8 mm to 10 mm along the edge of the incision 50, but the size of the incision 50 or the incision method is not limited thereto.

Referring to FIGS. 3 and 4C, a part of the exposed surface of the sclera 3 by dissecting the conjunctiva and the Tenon’s tissue may be cut to form the scleral flap 40 of which one side is connected to the sclera 3 and the other side may be cut and lifted up (S2). In an embodiment, before the formation of the scleral flap 40, blood vessels on the sclera may be removed. The scleral flap 40 may be formed in the shape of a flat plate that is about a few mm in horizontal and vertical lengths, and those skilled in the art will easily understand that the shape shown in the drawings may be exaggerated for convenience of description. For example, the length of each side of the scleral flap 40 may be about 3 mm, and the thickness of the scleral flap 40 may be from 1 mm to 2 mm. However, the size and thickness of the scleral flap 40 are not limited to the disclosed numerical values.

In an embodiment, after the scleral flap 40 is formed, the sclera 3 beneath the scleral flap 40 may be treated by an antifibrotic agent (S3). For example, an impregnation material such as a sponge may be soaked in the antifibrotic agent such as dexamethasone, mitomycin-C (MMC), 5-fluorouracil (5-FU), triamcinolone (TA), anti-Vascular Endothelial Growth Factor (VEGF) drugs, Transforming Growth Factor (TGF)-beta inhibitors, and the impregnation material may be placed on the area beneath the scleral flap 40 for a predetermined time (for example, 2 minutes to 3 minutes) to perform antifibrotic treatment. After the antifibrotic treatment, the impregnation material may be removed and the area beneath the scleral flap 40 may be washed (S3).

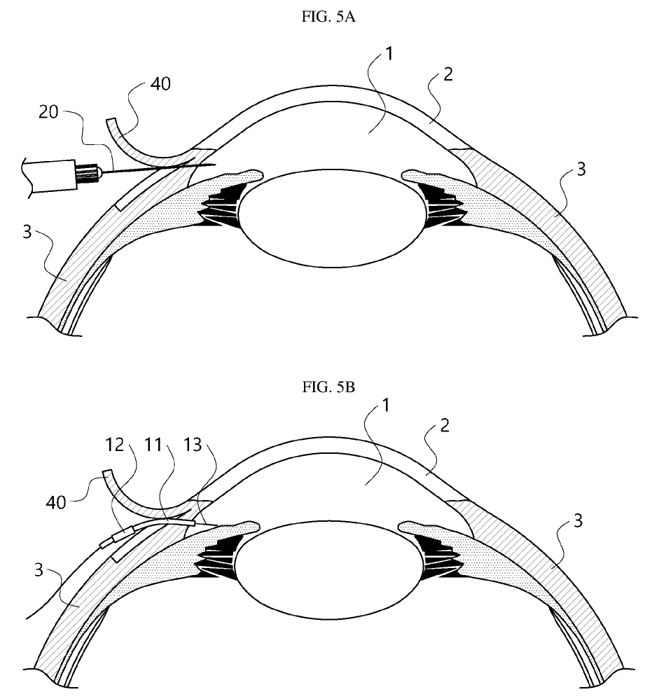

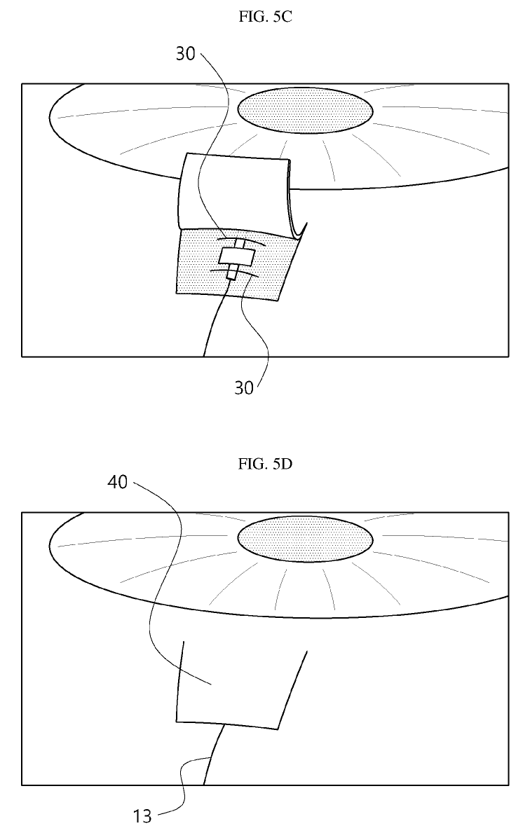

FIGS. 5A to 5D are conceptual views showing a process of inserting the implant under the scleral flap in the surgical method for insertion of the implant for eye diseases according to an embodiment.

Referring to FIGS. 3 and 5A, in an embodiment, anterior chamber paracentesis may be performed in the area beneath the scleral flap 40 lifted up from the eyeball before the implant insertion. This refers to a process of inserting a tool such as a syringe needle 20 into the sclera 3 under the scleral flap 40 to form a hole in the sclera 3. The location at which the hole is formed in the sclera 3 may be away from the edge of the cornea 2 by about a few mm (for example, 1 mm to 2 mm), but is not limited thereto.

Referring to FIGS. 3 and 5B, the tube 11 of the implant device may be inserted into the anterior chamber 1 of the eyeball through the hole formed in the sclera 3 beneath the scleral flap 40 (S4). The insertion of the tube 11 may be done through tweezers, an injector or any other appropriate tool. In this instance, the insertion location of the tube 11 may be determined such that one end of the tube 11 is located inside the anterior chamber 1 of the eyeball, and the other end is located outside of the sclera 3. Additionally, in an embodiment, the intraluminal stent 13 may be inserted inside the tube 11 to adjust the drainage volume of the aqueous humor. Meanwhile, the wing 12 extended in a direction that is different from the length direction of the tube 11 may be located outside of the sclera 3 and it may be possible to prevent the tube 11 from being completely inserted into the sclera 3 due to the presence of the wing 12.

Referring to FIGS. 3 and 5C, in an embodiment, suturing may be performed over the tube 11 of which one end is inserted into the sclera 3 with a suture thread 30 of nylon or Prolene (S5). The suturing plays a role in preventing the tube 11 from moving in the inward or outward direction of the eyeball 3 and fixing the position of the implant device.

Referring to FIGS. 3 and 5D, after the implant device is inserted into the eyeball under the scleral flap 40, the implant device may be covered over by the scleral flap 40 (S6). In an embodiment, after covering with the scleral flap 40, additional suturing may be performed to fix the position of the intraluminal stent 13 of the implant device exposed to the outside (S7), and it will be described in more detail. Subsequently, the conjunctiva that has been dissected may be sutured again, completing the surgery for insertion of the implant for eye diseases (S8).

In the surgical method according to the embodiments of the present disclosure as described above, one end of the implant device for eye diseases is inserted into the sclera under the scleral flap, and the other end is located outside of the sclera to drain the aqueous humor from the eye. In this instance, the end of the implant device located outside of the sclera is covered by the scleral flap, but in the surgical method, the generation of the scleral flap precedes the insertion to form the space between the aqueous humor outlet of the implant tube and the conjunctiva, thereby preventing direct contact between the implant tube and the Tenon’s capsule.

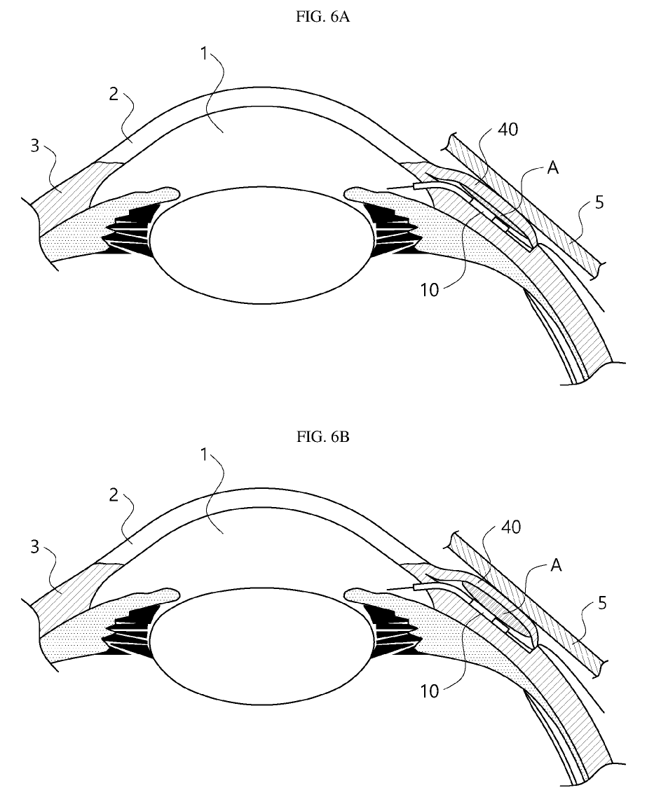

FIGS. 6A and 6B are conceptual views showing space formation between the aqueous humor outlet of the implant and the conjunctiva in the surgical method for insertion of the implant for eye diseases according to an embodiment.

Referring to FIG. 6A, in the surgical method according to the embodiments of the present disclosure, the implant device 10 is inserted into the eyeball under the scleral flap 40, but suturing that leads to blockage or narrowing of the route of aqueous humor drainage is not performed in the scleral flap 40 through the implant device 10. Conventionally, the scleral flap 40 covering the end portion of the implant device 10 exposed to the outside of the sclera 3 is completely sutured to the eyeball, but in the embodiments of the present disclosure, suturing of the scleral flap 40 is not performed, or if so, suturing of the scleral flap 40 is performed such that a side of the scleral flap 40 covering the tube exposed to the outside of the eyeball is lifted up. As a result, a space A by the aqueous humor drainage is formed at the aqueous humor outlet of the implant device 10, and with the increasing drainage volume of the aqueous humor, the size of the corresponding space A may increase as shown in FIG. 6B.

When the existing implant device is inserted into the eyeball, fibrosis usually occurs in the Tenon’s tissue between the sclera and the conjunctiva. However, by the surgical method according to the embodiments of the present disclosure, the aqueous humor outlet of the implant device 10 may be covered by the scleral flap 40 and the scleral flap 40 may be lifted up by the aqueous humor drainage through the implant device 10, so the aqueous humor forms the space A beneath the scleral flap 40. As a result, it may be possible to prevent contact between the aqueous humor outlet of the implant device 10 and the Tenon’s tissue, thereby preventing the aqueous humor outlet from being blocked by fibrosis and achieving continuous drainage of the aqueous humor through the implant device 10.

When the intraluminal stent inserted inside the tube of the implant device 10 is removed after a predetermined time (for example, about 4 weeks to 6 weeks) has passed since the implantation of the implant device 10, the drainage volume of the aqueous humor further increases, and the size of the space A formed through the aqueous humor drainage increases as shown in FIG. 6B, thereby preventing contact between the implant tube and the Tenon’s tissue.

That is, the surgical method according to the embodiments includes inserting the implant device 10 into the eyeball under the scleral flap 40 and covering the implant device 10 exposed to the outside of the eyeball by the scleral flap 40, wherein the scleral flap 40 is positioned such that a side of the scleral flap 40 covering the tube of the implant device 10 exposed to the outside of the eyeball is lifted up. As described above, after the implant device 10 is covered over by the scleral flap 40, suturing is not performed, or if so, suturing may be partially performed not to block or narrow the route of aqueous humor drainage through the implant device 10, so the scleral flap 40 may be positioned such that a side of the scleral flap 40 may be lifted up by the aqueous humor. For example, even though suturing of the scleral flap 40 is performed, the suturing is not performed in an area located further downstream than the aqueous humor outlet of the implant device 10 on the basis of the flow direction the aqueous humor draining from the scleral flap 40 through the implant device 10.

Meanwhile, in other embodiment, suturing of the scleral flap 40 may be performed such that a side of the scleral flap 40 covering the aqueous humor outlet of the implant device 10 may be folded up or bent up due to the presence of the suture thread.

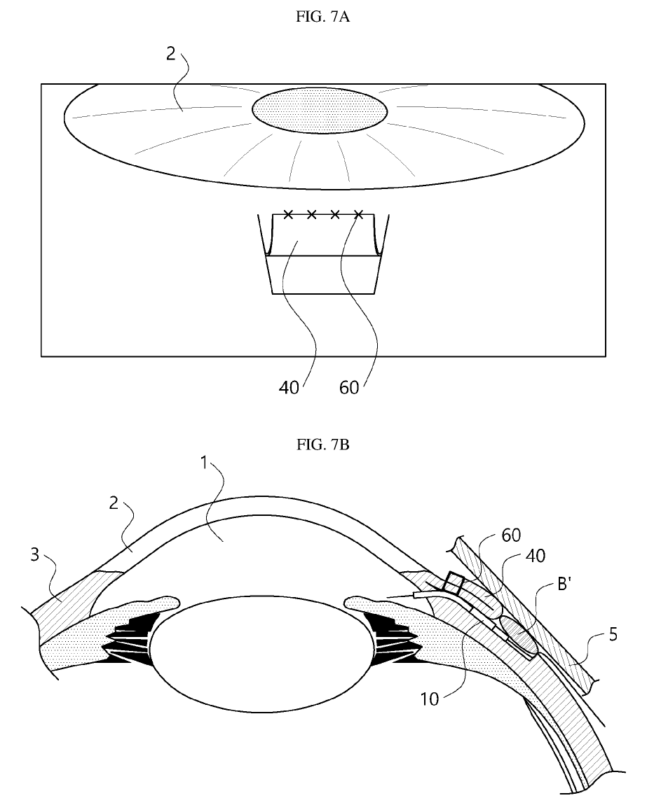

FIG. 7A is a conceptual view showing the scleral flap folded up in the surgical method for insertion of the implant for eye diseases according to an embodiment, and FIG. 7B is a cross-sectional view of FIG. 7A when viewed from the side of the eyeball.

Referring to FIGS. 7A and 7B, the surgical method according to this embodiment may include folding up a side of the scleral flap 40 that will cover the aqueous humor outlet of the implant device 10 when the scleral flap 40 stays still, and suturing the folded side with the suture thread (not shown) to the other portion of the scleral flap 40 or the eyeball to keep the scleral flap 40 in the folded state. In this instance, folding up refers to bending a part of the scleral flap 40 in a direction facing away from the eyeball.

As a result of folding up the scleral flap 40, a space B is formed with a height that is approximately double the thickness of the scleral flap 40 between the aqueous humor outlet of the implant device 10 and the conjunctiva 5. The physical space B may prevent direct contact between the tube of the implant device 10 and the Tenon’s tissue, thereby preventing the aqueous humor outlet of the implant device 10 from being blocked by fibrosis.

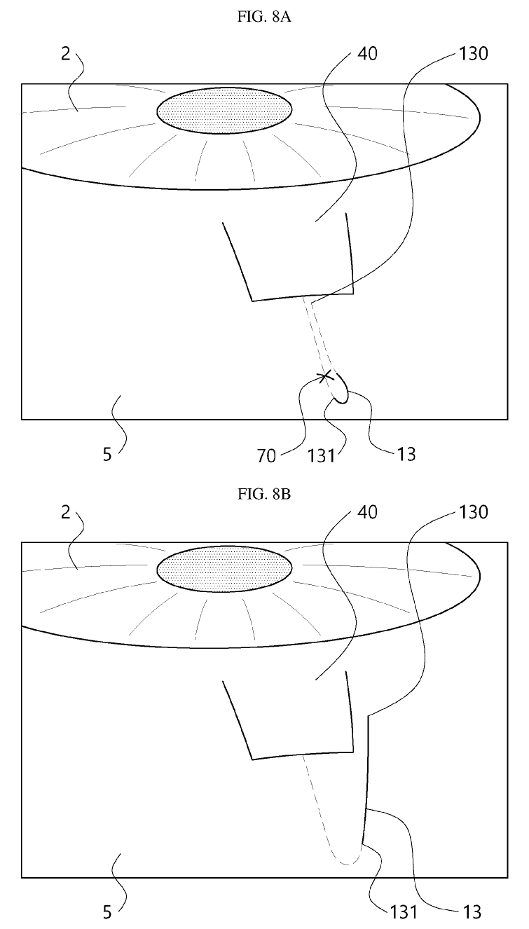

FIGS. 8A and 8B are conceptual views showing that the intraluminal stent exposed to the outside of the eyeball is secured using the conjunctiva in the surgical method for insertion of the implant for eye diseases according to an embodiment.

In the implant device covered by the scleral flap 40, the intraluminal stent 13 exposed through the proximal end of the tube may be extended outward from the covered area by the scleral flap 40. In this instance, some embodiments of the present disclosure may prevent the intraluminal stent 13 from moving or being buried at an unwanted location by allowing the intraluminal stent 13 exposed through the proximal end of the tube to pass through the conjunctiva 5 of the eyeball. More specifically, the position of the intraluminal stent 13 may be adjusted so that at least part of the intraluminal stent 13 exposed through the proximal end of the tube is exposed to the outside of the conjunctiva 5.

To this end, in an embodiment, referring to FIG. 8A, the position of the intraluminal stent 13 may be fixed in a way that the portion of the intraluminal stent 13 escapes from the eyeball through the conjunctiva 5, and the tip 130 of the intraluminal stent 13 is inserted into the conjunctiva 5 again. To place the tip 130 of the intraluminal stent 13 at a desired location, first, a syringe needle (not shown) may be inserted through the conjunctiva 5 down to the conjunctiva 5, and the tip 130 of the intraluminal stent 13 may be inserted into the syringe needle. Subsequently, when the syringe needle is moved out of the conjunctiva 5, the intraluminal stent 13 inserted inside the syringe needle is also pulled out of the conjunctiva 5. Subsequently, when the tip 130 of the intraluminal stent 13 is pushed into the conjunctiva 5 again by the same method, the intraluminal stent 13 may be secured such that a portion of the intermediate part of the intraluminal stent 13 is exposed to the outside of the conjunctiva 5 as shown in FIG. 8A.

In an embodiment, a process of suturing the intraluminal stent 13 to the sclera or the conjunctiva using the suture thread 70 may be additionally performed. In this instance, the location at which the suture thread 70 is coupled may be positioned between the rear boundary of the scleral flap 40 (that is, a part of the scleral flap 40 that is farthest away from the cornea 2) and a location 131 at which the intraluminal stent 13 escapes from the conjunctiva 5.

As another embodiment, referring to FIG. 8B, after the intraluminal stent 13 is pulled out of the conjunctiva 5 using the syringe needle as described above, the tip 130 of the intraluminal stent 13 may be exposed to the outside of the conjunctiva 5. In an embodiment, when the exposed portion of the intraluminal stent 13 to the outside of the conjunctiva 5 is too long, the exposed portion of the intraluminal stent 13 may be cut and removed. That is, the excessive intraluminal stent that is more posterior than the location 131 where the intraluminal stent 13 passes through the conjunctiva 5 to the outside may be cut to prevent the patient from feeling uncomfortable or foreign body sensation.

By the above-described embodiments of the present disclosure, the tube of the implant may be inserted into the eyeball under the scleral flap, and after the implant tube is inserted, the scleral flap may be positioned such that a side of the scleral flap may be lifted up to prevent the proximal end of the tube to drain the aqueous humor from being completely covered by the scleral flap, thereby preventing direct contact between the tube and the sclera and the Tenon’s capsule and preventing the aqueous humor outlet of the tube from being blocked by fibrosis. Additionally, the intraluminal stent exposed through the proximal end of the implant tube may be secured using the conjunctiva tissue, thereby preventing the intraluminal stent from slipping when the patient rubs the eye, and easily removing the intraluminal stent without affecting the aqueous humor drainage through the implant when removing the intraluminal stent after the predetermined time has passed since the implantation.

The above description of the present disclosure is provided for the purpose of illustration, and those skilled in the art will understand that the present disclosure can be easily modified in other particular forms without changing the technical spirit or essential features of the present disclosure. Therefore, it should be understood that the disclosed embodiments are provided by way of illustration, but not intended to be limiting, in all aspects. For example, each element described in singular forms may work in a distributed manner, and likewise, elements described in a distributed form may work in a combined manner.

The scope of the present disclosure is defined by the appended claims rather than the detailed description, and it should be interpreted that all changes or modifications derived from the meaning and scope of the appended claims and the equivalent concept are included in the scope of the present disclosure.

What is claimed is:

1. A surgical method for insertion of an implant for eye diseases, comprising:
cutting a portion of a sclera of an eyeball to form a scleral flap to be lifted up from the eyeball;
inserting an end of a tube including a hole for aqueous humor drainage from an anterior chamber of the eyeball into the eyeball through an area beneath the scleral flap; and
covering the tube exposed to outside of the eyeball with the scleral flap,
wherein an opposite end of the tube is not inserted into the eyeball, and
wherein covering with the scleral flap comprises placing the scleral flap on the tube without suturing of the scleral flap that leads to blockage or narrowing of a route of the aqueous humor drainage through the tube exposed to the outside of the eyeball.

2. The surgical method for insertion of the implant for eye diseases according to claim 1, wherein the covering with the scleral flap comprises:
folding the scleral flap such that a first side of the scleral flap covering the tube exposed to the outside of the eyeball is lifted up; and
suturing together a portion of the scleral flap and the eyeball or other portion of the scleral flap to prevent the folded scleral flap from being unfolded.

3. The surgical method for insertion of the implant for eye diseases according to claim 2, wherein the suturing comprises suturing together the first side of the scleral flap and a second side of the scleral flap opposite the first side, and
wherein an aqueous humor outlet of the tube is exposed, but not covered by the scleral flap.

4. The surgical method for insertion of the implant for eye diseases according to claim 1, wherein the tube further includes an intraluminal stent that is inserted into the hole and exposed through the opposite end of the tube, and
wherein the surgical method for insertion of the implant for eye diseases further comprises allowing, in at least part, the intraluminal stent exposed through the opposite end of the tube to pass through the conjunctiva of the eyeball to secure the intraluminal stent.

5. The surgical method for insertion of the implant for eye diseases according to claim 4, wherein the securing the intraluminal stent comprises exposing a portion of the intraluminal stent to outside of the conjunctiva of the eyeball.

6. The surgical method for insertion of the implant for eye diseases according to claim 5, wherein the securing the intraluminal stent further comprises inserting a tip of the intraluminal stent into the conjunctiva of the eyeball with the portion of the intraluminal stent staying outside of the conjunctiva of the eyeball.

7. The surgical method for insertion of the implant for eye diseases according to claim 5, wherein the securing the intraluminal stent further comprises suturing the intraluminal stent to the conjunctiva at a location between an area in which the portion of the intraluminal stent passes through the conjunctiva and a boundary of the scleral flap in order to secure the intraluminal stent.

8. The surgical method for insertion of the implant for eye diseases according to claim 5, wherein the exposing the portion of the intraluminal stent to the outside of the conjunctiva of the eyeball comprises:
inserting a syringe needle into the conjunctiva from the outside of the conjunctiva of the eyeball;
inserting the portion of the intraluminal stent into the syringe needle; and
removing the syringe needle from the conjunctiva to move the portion of the intraluminal stent out of the conjunctiva.

9. The surgical method for insertion of the implant for eye diseases according to claim 8, wherein the securing the intraluminal stent further comprises removing, in at least part, the intraluminal stent exposed to the outside of the conjunctiva of the eyeball.

10. The surgical method for insertion of the implant for eye diseases according to claim 1, further comprising, before inserting into the eyeball, treating the area beneath the scleral flap with an antifibrotic agent.

11. The surgical method for insertion of the implant for eye diseases according to claim 1, wherein the tube includes at least one wing extended in a direction that is different from a length direction of the tube and configured to limit an insertion location of the tube, and
wherein the inserting into the eyeball comprises adjusting the insertion location of the tube to prevent the at least one wing from being inserted into the eyeball.

12. The surgical method for insertion of the implant for eye diseases according to claim 1, wherein the inserting into the eyeball comprises:
forming a hole passing through the sclera at a location away from a connected part of the scleral flap to the sclera by a preset distance; and
inserting the end of the tube into the anterior chamber of the eyeball through the hole.
